(19) United States
(12) Patent Application Publication
(10) Pub. No.: US 2025/0295399 A1
(43) Pub. Date: Sep. 25, 2025

(54) LOCKING MECHANISM AND PROTRUSION LENGTH CONTROL FOR INTEGRATED DILATOR AND PERFORATION DEVICE

(71) Applicant: Boston Scientific Scimed, Inc., Maple Grove, MN (US)

(72) Inventors: Lauren Koon, Mississauga (CA); Christian Balkovec, Cambridge (CA); Alison Henstock, Milton (CA)

(21) Appl. No.: 19/086,948

(22) Filed: Mar. 21, 2025

Related U.S. Application Data
(60) Provisional application No. 63/568,961, filed on Mar. 22, 2024.

(51) Int. Cl. A61B 17/00

(52) U.S. Cl. CPC A61B 17/00234; A61B 2017/00247

(57) ABSTRACT

An access system for perforating a tissue within a heart is disclosed. The system includes a perforation device having a perforation device body including a proximal portion and a distal portion terminating in a distal tip. The system further includes a dilator comprising an elongated body, a proximal end and a distal end portion terminating in a distal tip, a handle connected to the proximal end of the elongated body and a dilator lumen extending through the handle and the elongated body and dimensioned to slidingly receive the perforation device. The system also includes an indicator mechanism positioned on the dilator indicating a protrusion length of the perforation device. Furthermore, the system includes a locking mechanism located on the dilator having an unlocked position and a locked position. The locking mechanism is configured to impede longitudinal movement of the perforation device within the dilator lumen.

### CROSS REFERENCE TO RELATED APPLICATIONS

This application claims priority to U.S. Provisional Patent Application No. 63/568,961 entitled “LOCKING MECHANISM AND PROTRUSION LENGTH CONTROL FOR INTEGRATED DILATOR AND PERFORATION DEVICE,” filed Mar. 22, 2024, which is hereby incorporated by reference in its entirety.

### TECHNICAL FIELD

The present disclosure relates to medical systems and methods for puncturing a tissue within a heart of a patient. More specifically, the present disclosure relates to a protrusion length indicator and locking mechanism for a perforation device integrated with a dilator.

### BACKGROUND

In general, commercially available devices for integrated needle-based perforation and dilation within a heart are cumbersome and difficult to control for a user. During tissue puncture of a heart, a metal puncture needle is integrated with, and protrudes from, an insulative dilator. The puncture needle may protrude from the dilator via a spring tensioned advancement point on the handle of the device. In this circumstance, a user must maintain forward pressure on an advancement point to maintain puncture needle protrusion and prevent it from retracting into the insulative dilator.

When performing a mechanical puncture with the metal puncture needle, full protrusion of the puncture needle may be advantageous, but advancement of the puncture needle against tissue exerts additional mechanical force and increases the likelihood of accidental mechanical puncture to unintended tissue sites. Conversely, when performing radiofrequency (RF) or electrical current-based tissue puncture, restricting the protrusion of the metal puncture needle to increase the current density of the metal puncture needle and reduce current leakage into the surrounding blood pool is more advantageous for puncture efficacy as a greater proportion of current is delivered into the tissue. Thus, precision in the protrusion length of the puncture needle, in both mechanical punctures and RF or electrical current-based tissue punctures, is important.

Furthermore, when performing electrical current-based tissue puncture, it is advantageous to prevent exposure of the full bevel of the metal puncture needle in cases where the metal puncture needle has an open lumen. By partially covering the open lumen, tissue coring is prevented when electrical current-based puncture is performed. Tissue coring occurs when a discrete piece of tissue is separated from the target puncture site and can cause embolism if released into the blood stream. Currently, however, there are no means for a user to precisely control or obtain feedback regarding the protrusion length of the metal puncture needle that is integrated within a dilator.

Moreover, during tissue puncture of a patient's heart, a user must simultaneously maintain position of the device on the septum, maintain dilator and sheath alignment, apply and maintain pressure to the puncture needle, apply forward pressure to puncture and dilate the septum, and optionally, apply electrocautery which requires a second person to activate a switch. During this procedure, operators have been known to cross their hands using the current workflow. A risk associated with the current design includes, but is not limited to, a suboptimal transseptal puncture location which increases procedure complexities.

Current dilators with integrated puncture needles are cumbersome to use due to the ergonomics and required steps of a transseptal puncture, as explained above. In current commercially available devices, the operator must apply and maintain pressure to extend and maintain protrusion of the puncture needle. Application of the pressure using the right thumb is burdensome due to the competing force needed to maintain position of the dilator hub with the remainder of the right hand. Common practice for operators includes maintaining contact and control of the sheath hub with the left hand.

In addition, current practices require the single operator to advance the dilator with the needle still protruded to gain left heart access. This step risks tamponade if the transseptal assembly jumps forward during crossing with the needle protruded. Tamponade is a medical emergency that takes place when abnormal amounts of fluid accumulate in the pericardial sac compressing the heart and leading to a decrease in cardiac output and shock. However, if the needle is withdrawn prior to dilator crossing, the left heart access will be lost.

Against this background, there exists a continuing need in the industry to provide improved perforation devices and methods to gain access to and puncture a tissue within a patient's heart. An object of the present invention is therefore to provide such an apparatus.

### SUMMARY

In Example 1, an access system for perforating a tissue within a heart includes a perforation device having a perforation device body including a proximal portion and a distal portion terminating in a distal tip. The access system also includes a dilator comprising an elongated body having a body length, a proximal end and a distal end portion terminating in a distal tip, a handle having a handle length and connected to the proximal end of the elongated body, and a dilator lumen extending through the handle and the elongated body and dimensioned to slidingly receive the perforation device, wherein the body length and the handle length together define a dilator length. The access system also includes an indicator mechanism positioned on the dilator indicating a protrusion length of the perforation device. The access system also includes a locking mechanism located on the dilator having an unlocked position and a locked position, wherein in the locked position the locking mechanism is in physical communication with the perforation device, and wherein the locking mechanism is configured to impede longitudinal movement of the perforation device within the dilator lumen.

Example 2 is the system of Example 1 further comprising a needle advancement mechanism positioned on the dilator.

Example 3 is the system of Example 2 wherein the needle advancement mechanism is configured to interact with the perforation device and advance the perforation device longitudinally within the dilator lumen.

Example 4 is the system of Example 3 wherein the perforation device includes at least one or more grooves located on the perforation device body, wherein the needle advancement mechanism is a knob having a top portion protruding from the dilator and a bottom portion positioned within the dilator lumen, and wherein when the knob aligns with one of the at least one or more grooves within the perforation device body, a user longitudinally advances the top portion of the knob across the dilator thereby advancing the perforation device longitudinally within the dilator lumen.

Example 5 is the system of Example 3 wherein the needle advancement mechanism is a dial having a traction element, and wherein friction applied by the dial to the perforation device advances the perforation device longitudinally within the dilator lumen.

Example 6 is the system of Example 2 wherein the needle advancement mechanism is an accessory device.

Example 7 is the system of Example 1 wherein the protrusion length is measured from the distal tip of the dilator to the distal tip of the perforation device.

Example 8 is the system of Example 1 wherein the indicator mechanism includes an indicator, and wherein the indicator is at least one of a visual, a tactile or an auditory indicator.

Example 9 is the system of Example 8 wherein the indicator includes at least one or more markings on the handle of the dilator representing the protrusion length of the perforation device.

Example 10 is the system of Example 8 wherein the indicator includes an indicator light that communicates the protrusion length of the perforation device.

Example 11 is the system of Example 1 wherein the locking mechanism includes a friction pad on a portion of the locking mechanism in contact with the perforation device, and wherein in the locked position, the pressure applied by the friction pad creates resistance on the perforation device thereby impeding longitudinal movement of the perforation device.

Example 12 is the system of Example 1 wherein the perforation device includes at least one or more grooves positioned on the perforation device body, and wherein in the locked position the locking mechanism aligns with one of the at least one or more grooves on the perforation device body to impede longitudinal movement of the perforation device.

Example 13 is the system of any of Examples 11-12 wherein the locking mechanism is a screw inserted into the handle of the dilator, the screw having a top portion protruding from the handle and a bottom portion positioned within the dilator lumen, wherein the top portion of the screw includes a wheel or a lever to be manipulated by a user, and wherein in the locked position the screw is in physical communication with the perforation device and impedes longitudinal movement of the perforation device.

Example 14 is the system of any of Examples 11-12 wherein the locking mechanism is a collar, and wherein in the locked position the collar tightens around the perforation device to impede longitudinal movement of the perforation device.

Example 15 is the system of any of Examples 11-12 wherein the locking mechanism is a spring positioned within the handle of the dilator, the spring having a top portion protruding from handle and a bottom portion positioned within the dilator lumen, wherein in the locked position the spring is in a neutral state and in physical communication with the perforation device, and wherein in the unlocked position the spring is in a compressed state and not in physical communication with the perforation device.

In Example 16, an access system for perforating a tissue within a heart includes a perforation device having a perforation device body including a proximal portion and a distal portion terminating in a distal tip. The access system also includes a dilator comprising an elongated body having a body length, a proximal end and a distal end portion terminating in a distal tip, a handle having a handle length and connected to the proximal end of the elongated body, and a dilator lumen extending through the handle and the elongated body and dimensioned to slidingly receive the perforation device, wherein the body length and the handle length together define a dilator length. The access system also includes an indicator mechanism positioned on the dilator indicating a protrusion length of the perforation device. The access system also includes a locking mechanism located on the dilator having an unlocked position and a locked position, wherein in the locked position the locking mechanism is in physical communication with the perforation device, and wherein the locking mechanism is configured to impede longitudinal movement of the perforation device within the dilator lumen.

Example 17 is the system of Example 16 further comprising a needle advancement mechanism positioned on the dilator, wherein the needle advancement mechanism is configured to interact with the perforation device and advance the perforation device longitudinally within the dilator lumen.

Example 18 is the system of Example 17 wherein the perforation device includes at least one or more grooves located on the perforation device body, wherein the needle advancement mechanism is a knob having a top portion protruding from the dilator and a bottom portion positioned within the dilator lumen, and wherein when the knob aligns with one of the at least one or more grooves within the perforation device body, a user longitudinally advances the top portion of the knob across the dilator thereby advancing the perforation device longitudinally within the dilator lumen.

Example 19 is the system of Example 17 wherein the needle advancement mechanism is a dial having a traction element, and wherein friction applied by the dial to the perforation device advances the perforation device longitudinally within the dilator lumen.

Example 20 is the system of Example 17 wherein the needle advancement mechanism is an accessory device.

Example 21 is the system of Example 16 wherein the protrusion length is measured from the distal tip of the dilator to the distal tip of the perforation device.

Example 22 is the system of Example 16 wherein the indicator mechanism includes an indicator, and wherein the indicator is at least one of a visual, a tactile or an auditory indicator.

Example 23 is the system of Example 22 wherein the indicator includes at least one or more markings on the handle of the dilator representing the protrusion length of the perforation device.

Example 24 is the system of Example 22 wherein the indicator includes an indicator light that communicates the protrusion length of the perforation device.

Example 25 is the system of Example 16 wherein the locking mechanism includes a friction pad on a portion of the locking mechanism in contact with the perforation device, and wherein in the locked position, the pressure applied by the friction pad creates resistance on the perforation device thereby impeding longitudinal movement of the perforation device.

Example 26 is the system of Example 16 wherein the perforation device includes at least one or more grooves positioned on the perforation device body, and wherein in the locked position the locking mechanism aligns with one of the at least one or more grooves on the perforation device body to impede longitudinal movement of the perforation device.

In Example 27, an access device for perforating a tissue within a heart includes a perforation needle having a perforation needle body including a proximal portion and a distal portion terminating in a distal tip. The access device also includes a dilator comprising an elongated body having a body length, a proximal end and a distal end portion terminating in a distal tip, a handle having a handle length and connected to the proximal end of the elongated body, and a dilator lumen extending through the handle and the elongated body and dimensioned to slidingly receive the perforation device, wherein the body length and the handle length together define a dilator length. The access device also includes an indicator mechanism positioned on the dilator indicating a protrusion length of the perforation needle. The access device also includes a needle advancement mechanism positioned on the dilator configured to move the perforation needle longitudinally within the dilator lumen. The access device also includes a locking mechanism located on the dilator having an unlocked position and a locked position, wherein in the locked position the locking mechanism is in physical communication with the perforation needle, and wherein the locking mechanism is configured to impede longitudinal movement of the perforation needle within the dilator lumen.

Example 28 is the device of Example 27 wherein the protrusion length is measured from the distal tip of the dilator to the distal tip of the perforation device.

Example 29 is the device of Example 27 wherein the indicator mechanism includes an indicator, and wherein the indicator is at least one of a visual, a tactile or an auditory indicator.

Example 30 is the device of Example 27 wherein the locking mechanism includes a friction pad on a portion of the locking mechanism in contact with the perforation device, and wherein in the locked position, the pressure applied by the friction pad creates resistance on the perforation device thereby impeding longitudinal movement of the perforation device.

Example 31 is the device of Example 27 wherein the perforation device includes at least one or more grooves positioned on the perforation device body, and wherein in the locked position the locking mechanism aligns with one of the at least one or more grooves on the perforation device body to impede longitudinal movement of the perforation device.

Example 32 is the device of Example 27 wherein the locking mechanism is a screw inserted into the handle of the dilator, the screw having a top portion protruding from the handle and a bottom portion positioned within the dilator lumen, wherein the top portion of the screw includes a wheel or a lever to be manipulated by a user, and wherein in the locked position the screw is in physical communication with the perforation device and impedes longitudinal movement of the perforation device.

Example 33 is the device of Example 27 wherein the locking mechanism is a collar, and wherein in the locked position the collar tightens around the perforation device to impede longitudinal movement of the perforation device.

Example 34 is the device of Example 27 wherein the locking mechanism is a spring positioned within the handle of the dilator, the spring having a top portion protruding from handle and a bottom portion positioned within the dilator lumen, wherein in the locked position the spring is in a neutral state and in physical communication with the perforation device, and wherein in the unlocked position the spring is in a compressed state and not in physical communication with the perforation device.

In Example 35, a method for perforating a tissue within a heart includes providing a perforation device having a perforation device body including a proximal portion and a distal portion terminating in a distal tip. The method for perforating a tissue within a heart also includes providing a dilator comprising an elongated body having a body length, a proximal end and a distal end portion terminating in a distal tip, a handle having a handle length and connected to the proximal end of the elongated body, and a dilator lumen extending through the handle and the elongated body and dimensioned to slidingly receive the perforation device, wherein the body length and the handle length together define a dilator length. The method for perforating a tissue within a heart also includes advancing an indicator mechanism positioned on the dilator indicating a protrusion length of the perforation device. The method for perforating a tissue within a heart also includes using a locking mechanism located on the dilator having an unlocked position and a locked position, wherein in the locked position the locking mechanism is in physical communication with the perforation device, and wherein the locking mechanism is configured to impede longitudinal movement of the perforation device within the dilator lumen.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIGS. 1A-1B are schematic illustrations of a medical procedure within a patient's heart for gaining access to the transseptal access as well as access to the epicardial space, according to embodiments of the present disclosure.

FIG. 2 is an illustration of an access system for perforating a tissue within the patient's heart having a dilator and a perforation device, according to embodiments of the present disclosure.

FIGS. 3A-3C are illustrations of an indicator mechanism and a needle advancement mechanism integrated with the dilator and the perforation device of FIG. 2, according to embodiments of the present disclosure.

FIGS. 4A-4F are schematic illustrations of a locking mechanism, integrated with the dilator and the perforation device of FIG. 2, including a screw, according to embodiments of the present disclosure.

FIGS. 5A-5D are schematic illustrations of a locking mechanism, integrated with the dilator and the perforation device of FIG. 2, including a screw collar having a ring or a collar positioned around the perforation device, according to embodiments of the present disclosure.

FIGS. 6A-6E are schematic illustrations of a locking mechanism, integrated with the dilator and the perforation device of FIG. 2, including a piston having a wheel or an axle, according to embodiments of the present disclosure.

FIGS. 7A-7F are schematic illustrations of a locking mechanism, integrated with the dilator and the perforation device of FIG. 2, including a clamp collar positioned around the perforation device, according to embodiments of the present disclosure.

FIGS. 8A-8F are schematic illustrations of a locking mechanism, integrated with the dilator and the perforation device of FIG. 2, including a torsion spring, according to embodiments of the present disclosure.

FIGS. 9A-9C are schematic illustrations of a locking mechanism, integrated with the dilator and the perforation device of FIG. 2, including a lifting clamp having a locking feature, according to embodiments of the present disclosure.

FIGS. 10A-10C are schematic illustrations of a locking mechanism, integrated with the dilator and the perforation device of FIG. 2, including a twist lock feature, according to embodiments of the present disclosure.

FIGS. 11A-11B are schematic illustrations of a locking mechanism, integrated with the dilator and the perforation device of FIG. 2, including a spring having a locking feature, according to embodiments of the present disclosure.

FIGS. 12A-12B are schematic illustrations of a locking mechanism, integrated with the dilator and the perforation device of FIG. 2, including a pumping feature connected to a balloon, according to embodiments of the present disclosure.

FIGS. 13A-13H are schematic illustrations of a locking mechanism, integrated with the dilator and the perforation device of FIG. 2, including a magnetic feature, according to embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

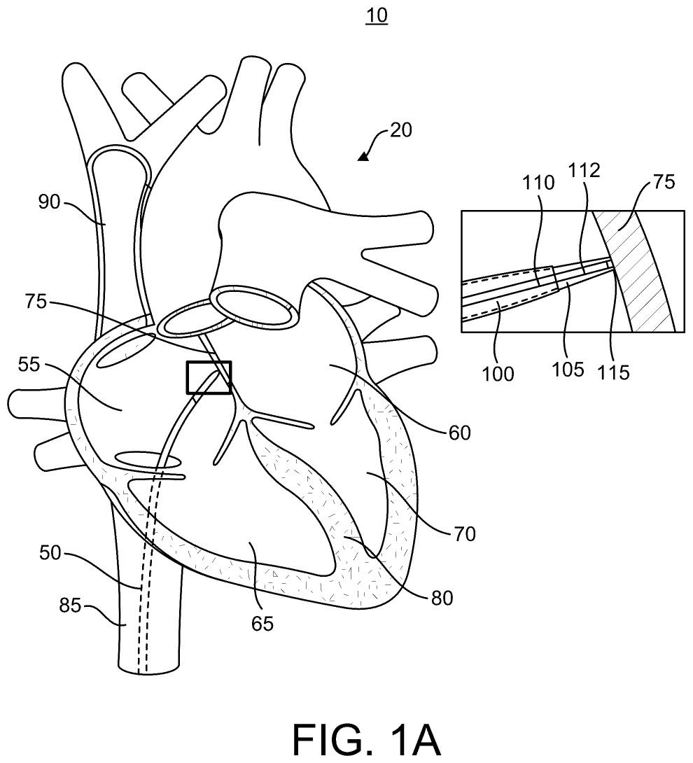

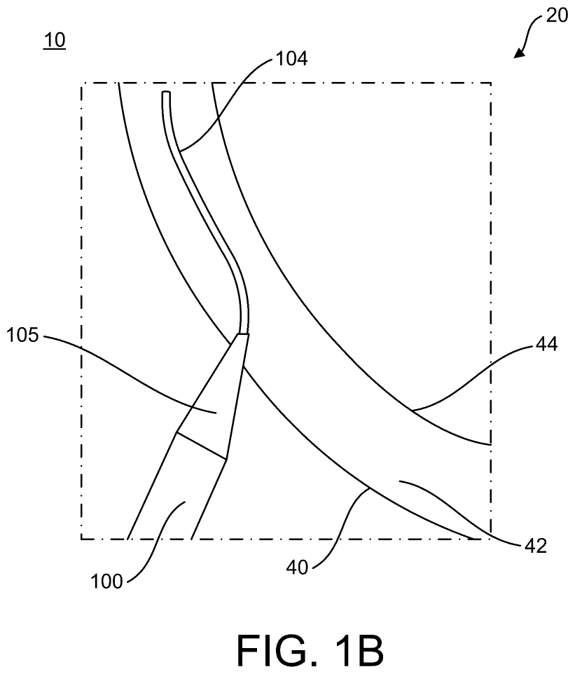

FIGS. 1A and 1B are schematic illustrations of a medical procedure within a patient's heart for gaining transseptal access as well as access to the epicardial space, according to embodiments of the present disclosure. FIG. 1A is an illustration of a medical procedure 10 within a patient's heart 20 utilizing a transseptal access system 50. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

Procedures for providing access to the left atrium 60 use transseptal access systems and devices for subsequent deployment of the aforementioned diagnostic and/or therapeutic devices within the left atrium 60. In these procedures, a target tissue site can be defined by tissue on the atrial septum 75. The target site is accessed via the inferior vena cava (IVC) 85, for example through the femoral vein, according to conventional catheterization techniques. In other embodiments, access to the target site on the atrial septum 75 may be accomplished using a superior approach wherein the transseptal access system 50 is advanced into the right atrium 55 via the superior vena cava (SVC) 90.

Transseptal access system procedures may include many devices like an introducer sheath 100, a dilator 105, a puncture device 110 having distal end portion 112 terminating in a tip electrode 115, and a guidewire. In various embodiments, the puncture device 110 is a mechanical puncture device (e.g., a needle) or an RF perforation device. The puncture device 110 can be disposed within the dilator 105, which itself can be disposed within the sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20.

When the guidewire, sheath 100 and dilator 105 have been positioned in the SVC 90, the guidewire is removed from the body, and the sheath 100 and the dilator 105 are retracted so that their distal ends are positioned in the right atrium 55. In an embodiment, the puncture device 110 described can then be introduced into the dilator 105, and advanced toward the heart 20. In other embodiments, the puncture device 110 described may be introduced prior to the retraction of the sheath 100 and the dilator 105 from the SVC into the right atrium 55. The puncture device 110 is then positioned such that the tip electrode 115 is aligned with or protruding slightly from the distal end of the dilator 105. In embodiments where the puncture device 110 is an RF perforation device, with the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least 200 V RMS, or in certain embodiments about 565 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 0.1 second and about 5 seconds. In other embodiments, the step of delivering energy occurs over a period of about 300 milliseconds.

Still another medical procedure 10 developed for diagnosing or treating physiological ailments originating within a heart 20 includes epicardial ablation to help restore a regular heart rhythm, as shown in FIG. 1B. As illustrated, the heart 20 includes a pericardium 40, a pericardial cavity 42 and a myocardium 44. The heart 20 is typically approached using a subxiphoid approach. Epicardial access is achieved via puncturing a layer of the pericardium 40 while avoiding the myocardium 44 of the heart. The pericardium 40 is a tough, double-walled, fibroelastic sac encompassing the heart 20 and the roots of the great vessels. The pericardium 40 includes two layers, an outer layer made of strong connective tissue often referred to as the fibrous pericardium, and an inner layer made of serous membrane often referred to as the serous pericardium. The mesothelium, or mesothelial cells, that constitutes the serous pericardium also covers the myocardium of the heart as epicardium, resulting in a continuous serous membrane invaginated onto itself as two opposing surfaces such as over the fibrous pericardium 40 and over the heart 20. This creates a pouch-like virtual or potential space around the heart 20 enclosed between the two opposing serosal surfaces, often referred to as the pericardial space or pericardial cavity 42.

In some embodiments, the pericardium 40 may be punctured with a puncture device 110, such as a needle (or other mechanical puncture device). Once punctured, a dilator 105 is advanced to dilate the puncture created by the needle through the pericardium 40. In certain embodiments, a sheath 100 may be advanced with the dilator 105 simultaneously. In other embodiments, the sheath 100 may be advanced afterwards. The sheath 100 and the dilator 105 may then be withdrawn to leave the guidewire 104 in the pericardial cavity 42. Minimally invasive access to the epicardium is required for diagnosis and treatment of a variety of arrhythmias and other conditions. During epicardial ablation, tiny scars are created on the outside of the heart to create a transmural lesion. In other words, to achieve an ablated tissue through the thick muscle of the heart.

The present disclosure describes novel systems and methods for providing safe access to the heart. As will be explained in greater detail herein, the embodiments of the present disclosure improve the means of identifying the protrusion length of a puncture device that is integrated with a dilator within the heart of a patient and locking the puncture device at a specific protrusion length within the heart.

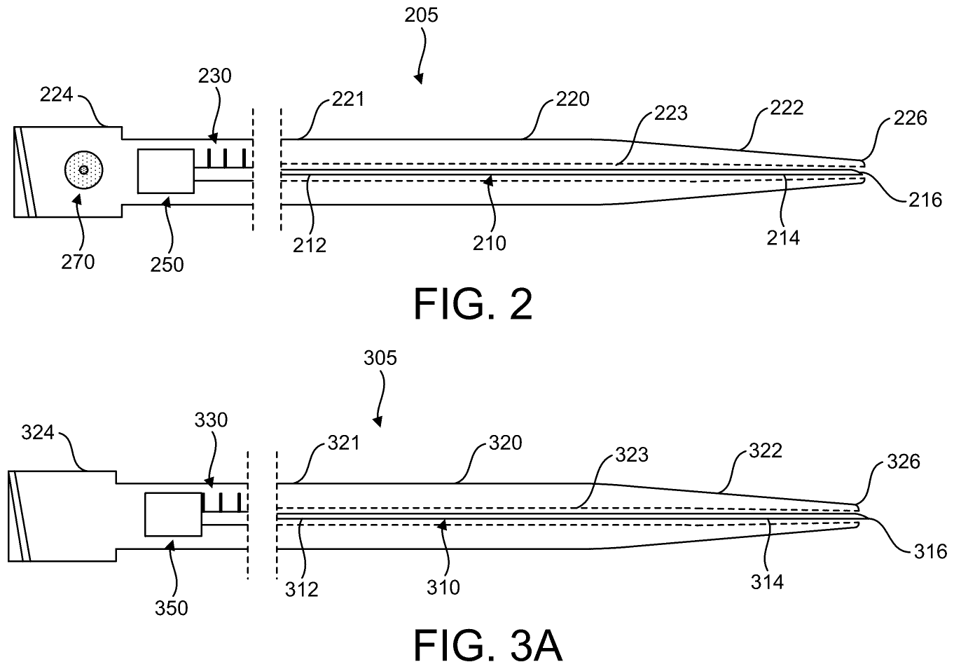

FIG. 2 is an illustration of an access system for perforating a tissue within a heart having a dilator 205 and a perforation device 210, according to an embodiment of the present disclosure. As shown, the dilator 205 includes an elongated dilator body 220 having a body length, a dilator handle 224 having a handle length, and a dilator lumen 223 extending longitudinally through the handle 224 and the dilator body 220. Additionally, the dilator body 220 includes a proximal end portion 221 and an opposite distal end portion 222 terminating in a distal tip 226. In an embodiment, the handle 224 is connected to the proximal end portion 221 of the dilator body 220. In an embodiment, the body length and the handle length together define a dilator length. Additionally, as shown, the lumen 223 of the dilator 205 is dimensioned to slidingly receive the perforation device 210. perforation device body having a proximal portion 212 and a distal portion 214 extending from the proximal portion 212 and terminating in a distal tip 216. In some embodiments, the perforation device 210 may be an RF perforation device. In certain embodiments, the distal tip 216 may include a distal tip electrode (e.g., a tip electrode such as described above in connection with FIGS. 1A-1B). In other embodiments, the perforation device 210 may be a mechanical perforation device. As will be appreciated, in some embodiments, the length of the perforation device 210 is greater than the length of the dilator 205 so that part of the proximal portion 212 of the perforation device 210 extends proximally of the handle 224 (not shown) when the distal portion 214, particularly the distal tip 216, extends distally of the dilator 205, thus allowing the proximal portion 212 to be manipulated by the user as needed.

In some embodiments, the proximal portion 212 of the perforation device 210 has an electrically insulated outer surface. As such, the proximal portion 212 can be handled directly by the user when the perforation device 210 is energized. In some embodiments, the proximal portion 212 is of a unitary construction formed entirely of an electrically insulative material. In certain embodiments, one exemplary class of materials for construction of the proximal portion 212 can include various grades of polytetrafluoroethylene (PTFE), polyetheretherketone (PEEK), among others. In some embodiments, the proximal portion 212 can further include reinforcing elements, e.g., a polymeric braid or coil, to enhance the structural properties, e.g., stiffness, torque transfer capability, and the like. In some embodiments, the proximal portion 212 is formed of a metal (e.g., a metal hypotube), and includes an outer electrically insulating layer.

In certain embodiments, the distal portion 214 is electrically conductive and is capable of transferring radiofrequency energy supplied by an external RF generator to the functional tip 216 for subsequent delivery to the target tissue in a transseptal crossing or an epicardial ablation procedure, as described above. Any biocompatible electrically conductive material may be selected for construction of the distal portion 214. Exemplary materials may include stainless steel, nickel-titanium alloy, and the like. Further, for ease of illustration, the distal portion 214 is depicted in FIG. 2 as a single solid structure, although the construction of the distal portion 214 can vary to accommodate the particular structural requirements for the perforation device 210, as will be further explained below. For example, in some embodiments, the distal portion 214 can be constructed as a solid rod, a tube or a coil.

Additionally, in certain embodiments, the distal portion 214 can be constructed in multiple segments, e.g., a solid rod or hypotube in the regions nearest the proximal portion 212, and a coiled structure more distally to provide enhanced flexibility and torqueability. In some embodiments, the distal portion 214 can have a composite construction, e.g., a solid or tubular core conductor surrounded by a wire coil. Additionally, as shown in the illustrated embodiment, the proximal and distal portions 212, 214 are substantially isodiametric, although this is not a strict requirement in all embodiments.

As further shown in FIG. 2, the access system includes an indicator mechanism 230 positioned on the dilator 205. In certain embodiments, the indicator mechanism 230 may be positioned on the dilator handle 224. In an embodiment, the indicator mechanism 230 defines a protrusion length of the perforation device 210. In some embodiments, the protrusion length is measured from the distal tip 226 of the dilator 205 to the distal tip 216 of the perforation device 210. Additionally, the access system further includes a locking mechanism 270 located on the dilator 205. In certain embodiments, the locking mechanism 270 may be positioned on the dilator handle 224, as shown. In an embodiment, as will be discussed in greater detail below, the locking mechanism 270 includes an unlocked position and a locked position, wherein in the locked position the locking mechanism 270 is in physical communication with the perforation device 210. Additionally, in an embodiment, the locking mechanism 270 is configured to impede longitudinal movement of the perforation device 210 within the dilator lumen 223. Furthermore, as shown in FIG. 2, in some embodiments, the access system for perforating a tissue within a heart of a patient may further comprise a needle advancement mechanism 250 positioned on the dilator 205. In certain embodiments, the needle advancement mechanism 250 may be positioned on the dilator handle 224, as shown.

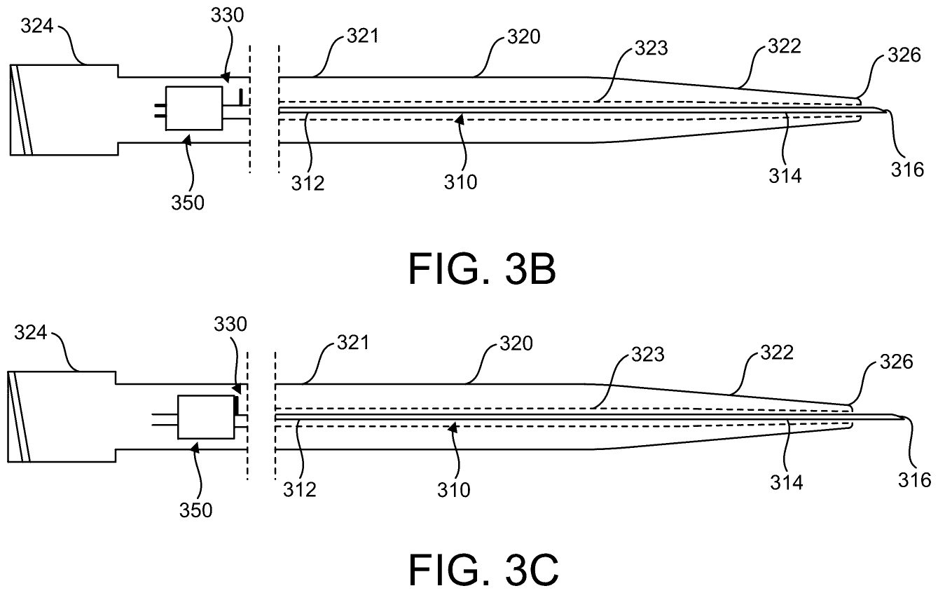

FIGS. 3A-3C are illustrations of an indicator mechanism 330 and a needle advancement mechanism 350 integrated with a dilator 305 and a perforation device 310, according to embodiments of the present disclosure. As shown, in some embodiments, the needle advancement mechanism 350 is configured to interact with the perforation device 310 and advance the perforation device 310 longitudinally within the dilator lumen 323. In certain embodiments, the needle advancement mechanism 350 may be positioned on the dilator handle 324, as shown. In some embodiments, the needle advancement mechanism 350 may be a knob, a dial, a screw, a collar or any other structure known in the art that may guide in advancing the perforation device 310.

In some embodiments, the perforation device 310 may include at least one or more grooves located on the body of the perforation device 310. Additionally, in some embodiments, the needle advancement mechanism 350 may be a knob, or a screw, having a top portion that protrudes from the dilator handle 324, so that a user may have access to the needle advancement mechanism 350, and a bottom portion positioned through the dilator handle 324 and within the dilator lumen 323. In certain embodiments, when the knob aligns with one of the at least one or more grooves within the perforation device body 310, a user may longitudinally advance the top portion of the knob across the dilator 305 thereby advancing the perforation device 310 longitudinally within the dilator lumen 323. Thus, in one embodiment, the perforation device 310 is selectively advanced by the needle advancement mechanism 350 on the dilator handle 324 of the integrated perforation device 310 and dilator 305.

In other embodiments, the needle advancement mechanism 350 may be a dial having a traction element, a top portion protruding from the dilator handle 324 that is accessible to the user and a bottom portion positioned through the dilator handle 324 and within the dilator lumen 323 having access to the perforation device 310. In certain embodiments, the friction applied by the dial to the perforation device 310 may advance the perforation device 310 longitudinally within the dilator lumen 323. Still other configurations known in the art may be contemplated to selectively advance the needle advancement mechanism 350 on the dilator handle 324 and thereby advancing the perforation device 310. In certain embodiments, the needle advancement mechanism 350 may be an accessory device that can be connected to the dilator 305 to aid in advancing the perforation device 310. In other embodiments, the dilator 305 may include the indicator mechanism 330 without the need for the needle advancement mechanism 350.

As shown in FIGS. 3A-3C, the access system includes an indicator mechanism 330 positioned on the dilator 305. In certain embodiments, the indicator mechanism 330 may be positioned on the dilator handle 324, as shown. In other embodiments, the indicator mechanism 330 may be positioned on the dilator body 320. In an embodiment, the indicator mechanism 330 indicates a protrusion length of the perforation device 310. Thus, in certain embodiments, the indicator mechanism 330 may provide a user with feedback regarding the protrusion length of the perforation device 310 from the dilator 305. In some embodiments, the protrusion length is measured from the distal tip 326 of the dilator 305 to the distal tip 316 of the perforation device 310. In some embodiments, the indicator mechanism 330 may indicate the length of the perforation device 310 from the dilator 305, while in other embodiments, the indicator mechanism 330 may indicate meaningful protrusion points along the perforation device 310, as will be discussed in greater detail below.

In certain embodiments, the indicator mechanism 330 includes an indicator that may be a visual, a tactile or an auditory indicator. In certain embodiments, other indicators known in the art that provide communication to the user regarding the protrusion length of the metal perforation device 310 may be contemplated. By explicitly indicating the amount of protrusion of the perforation device 310 from the dilator 305, the user may more precisely tailor the protrusion length of the perforation device 310 to optimize the perforation device method that is being employed.

In one embodiment, the indicator mechanism 330 may include an indicator light that communicates the protrusion length of the perforation device 310. In other embodiments, the indicator mechanism 330 may include at least one or more tactile notches, or raised surface features, that allow a user to feel and/or observe the position of the perforation device 310, and/or the advancement mechanism 350 on the dilator 305. In still other embodiments, the indicator mechanism 330 may include auditory feedback, such as clicking sounds, that are created when the user advances the perforation device 310 past certain points within the dilator 305. In certain embodiments, each indicator of the indicator mechanism 330 may correspond to one millimeter of advancement by the perforation device 310. In certain embodiments, each indicator of the indicator mechanism 330 may correspond to less than one millimeter of advancement by the perforation device 310. In certain embodiments, each indicator of the indicator mechanism 330 may correspond to two millimeters of advancement by the perforation device 310. In other embodiments, any other application of force or motion may be translated into the lateral movement of the perforation device 310.

As shown in FIGS. 3A-3C, in one embodiment, the indicator mechanism 330 may include at least one or more markings on the dilator handle 324 representing the protrusion length of the perforation device 310. Thus, in some embodiments, as the perforation device 310 is advanced longitudinally through the dilator lumen 323, whether by the needle advancement mechanism 350 or by any other means, the indicator mechanism 330 may decrease or increase in the markings presented which correlates to the protrusion length of the perforation device 310. In one example, as the protrusion length of the perforation device 310 increases from FIGS. 3A-3C, the markings of the indicator mechanism 330 decrease, depicting that the perforation device 310 protrusion length is increasing. In some embodiments, each marking on the dilator handle 324 may correspond to one millimeter of protrusion of the perforation device 310. Thus, in an embodiment, with each indicator marking reached, the perforation device 310 has been further advanced from the dilator 305. In certain embodiments, the indicators of the indicator mechanism 330 may correlate to meaningful protrusion points of the perforation device 310. For example, in one embodiment, the protrusion points may include no protrusion of the perforation device 310 (i.e., the perforation device 310 is inside the dilator 305), as shown in FIG. 2, the distal tip 316 of the perforation device 310 is protruded out of the dilator 305 slightly, as shown in FIG. 3A, and the perforation device 310 is fully protruded, as shown in FIG. 3C. As shown, in some embodiments, the indicator mechanism 330 indicates the amount of protrusion of the perforation device 310 from the dilator 305 such that the user may tailor the protrusion length of the perforation device 310 to optimize the puncture that is being employed.

As will be discussed below, in still other embodiments, a locking mechanism may be integrated with the dilator 305 to allow a user to fix the protrusion of the perforation device 310 from the dilator 305 without having to constantly maintain forward advancement pressure of the perforation device 310.

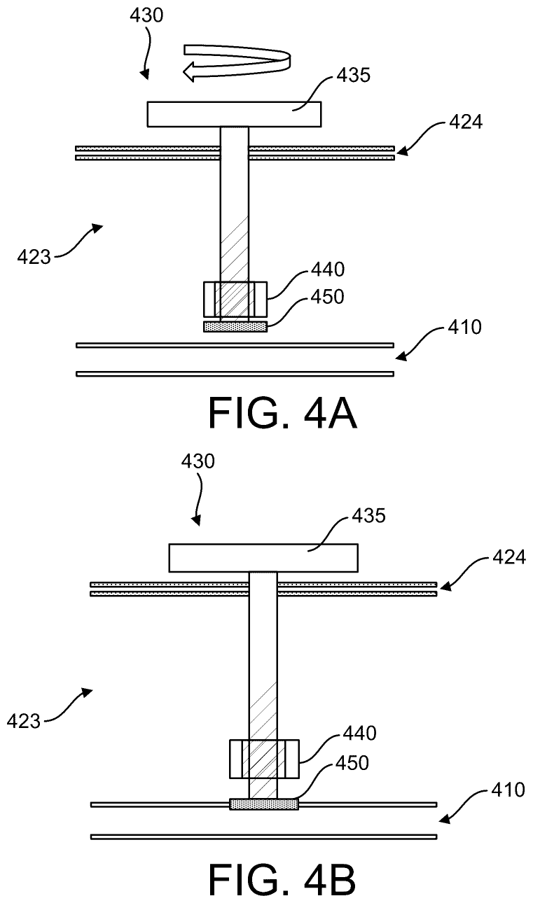

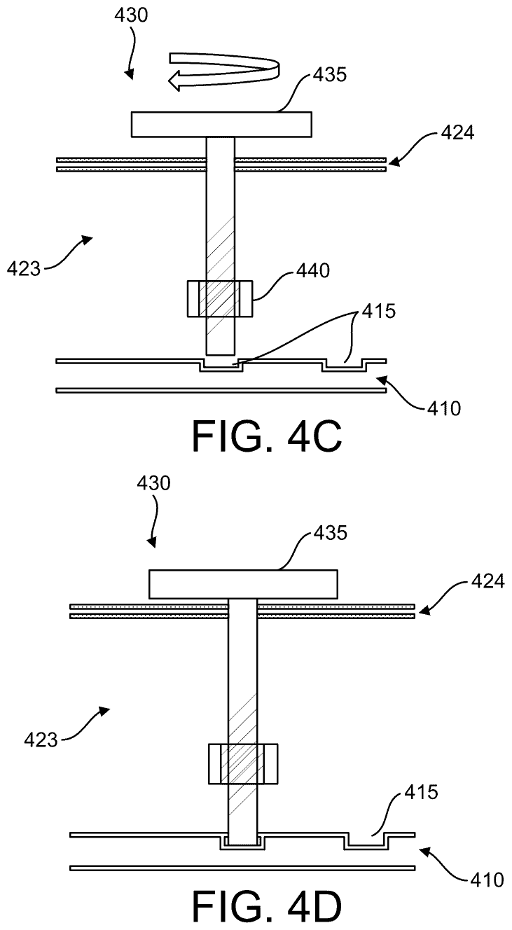

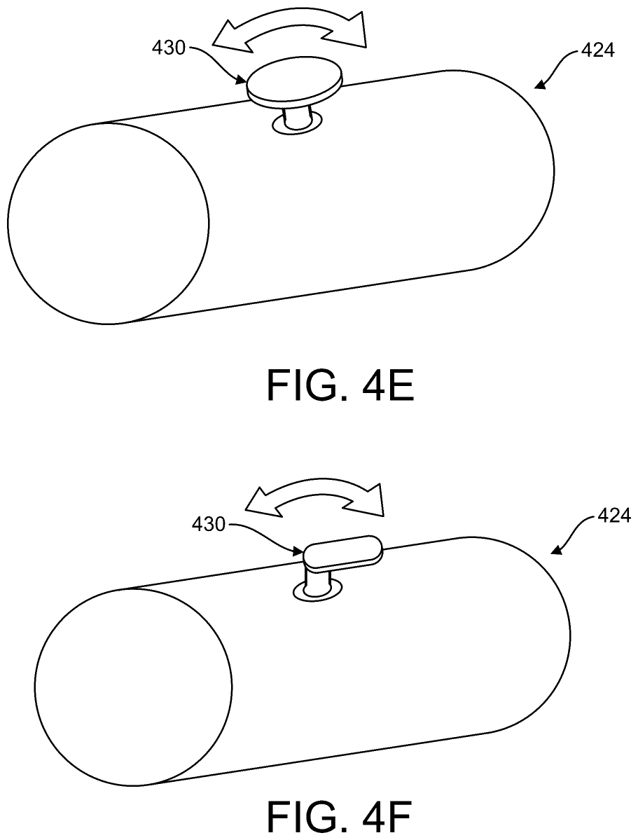

FIGS. 4A-13G are schematic illustrations of various locking mechanisms located on a dilator to impede longitudinal movement of a perforation device, according to embodiments of the present disclosure. In an embodiment, as shown in FIG. 2, an access system for perforating a tissue within a heart of a patient includes a locking mechanism located on the dilator having an unlocked position and a locked position. In some embodiments, in the unlocked position the locking mechanism is not in physical communication with the perforation device, while in the locked position the locking mechanism is in physical communication with the perforation device. In some embodiments, the locking mechanism is positioned on the dilator handle. Additionally, the locking mechanism is configured to impede longitudinal movement of the perforation device within the dilator lumen. In some embodiments, the locking mechanism may work congruently with the indicator mechanism and the needle advancement mechanism discussed above. Thus, in some embodiments, once the perforation device is advanced by the needle advancement mechanism and is positioned at the desired perforation length by the aid of the indicator mechanism, the perforation device may then be locked in position by the locking mechanism. In certain embodiments, the locking mechanism may include a screw mechanism, a collar mechanism, a piston mechanism, a clamp mechanism, a torsion spring mechanism, a lifting clamp mechanism, a twist lock mechanism, a spring mechanism, a pumping mechanism, a magnet mechanism, a combination thereof or any other mechanisms known in the art to impede longitudinal movement of the perforation device within the dilator lumen.

In some embodiments, as will be discussed in greater detail herein, the locking mechanism may include a friction pad on a portion of the locking mechanism, wherein in the locked position, the pressure applied by the friction pad onto the perforation device may create resistance on the perforation device thereby impeding longitudinal movement of the perforation device. In other embodiments, as will be discussed in greater detail herein, the perforation device may include at least one or more grooves positioned on the perforation device body and in the locked position the locking mechanism may align with of the at least one or more grooves on the perforation device body to impede longitudinal movement of the perforation device.

In some embodiments, as shown in FIGS. 4A-4F, the locking mechanism may include a screw mechanism 430 comprising a screw 435 having a top portion protruding from the dilator handle 424 and a bottom portion positioned within the dilator lumen 423. In some embodiments, the top portion of the screw 435 may include a wheel, as shown in FIG. 4E, or a lever, as shown in FIG. 4F, to be controlled by a user. In other embodiments, any structure known in the art used to control a screw by a user may be employed. In certain embodiments, the screw 435 may further comprise a nut 440 that aids in the twisting of the screw 435. In some embodiments, the nut 440 may be attached to the dilator handle 424. In one example, as depicted in FIGS. 4A-4B, the screw 435 may further include a friction pad 450 positioned on the most distal portion of the bottom portion of the screw 435. In some embodiments, as depicted, rotation of the screw 435 may raise or lower the screw 435 within the dilator lumen 423. As the screw 435 lowers within the dilator lumen 423, the locking mechanism 430 moves from an unlocked position, FIG. 4A, in which the screw 435 is not in contact with the perforation device 410 to a locked position, FIG. 4B, in which the screw 435, and more particularly the friction pad 450 of the screw 435, is in contact with the perforation device 410. Thus, in the locked position, once the screw 435 is in contact with the perforation device 410, by being lowered by the user, the friction pad 450 may hold the perforation device 410 in place, thereby impeding longitudinal movement of the perforation device 410. In some embodiments, the screw 435 may move back into the unlocked position by rotating the screw 435 upwards and removing the friction pad 450 from the perforation device 410.

In other embodiments, as shown in FIGS. 4C-4D, the perforation device 410 may include at least one or more grooves 415 positioned at set locations on the body of the perforation device 410. In some embodiments, as the screw 435 is lowered and moved to the locked position, the screw 435 may align with one of the at least one or more grooves 415 on the body of the perforation device 410 to impede longitudinal movement of the perforation device 410 at a defined location. In some embodiments, the screw 435 may move back to the unlocked position by raising the screw 435 upwards and taking the bottom portion of the screw 435 out of the at least one or more grooves 415.

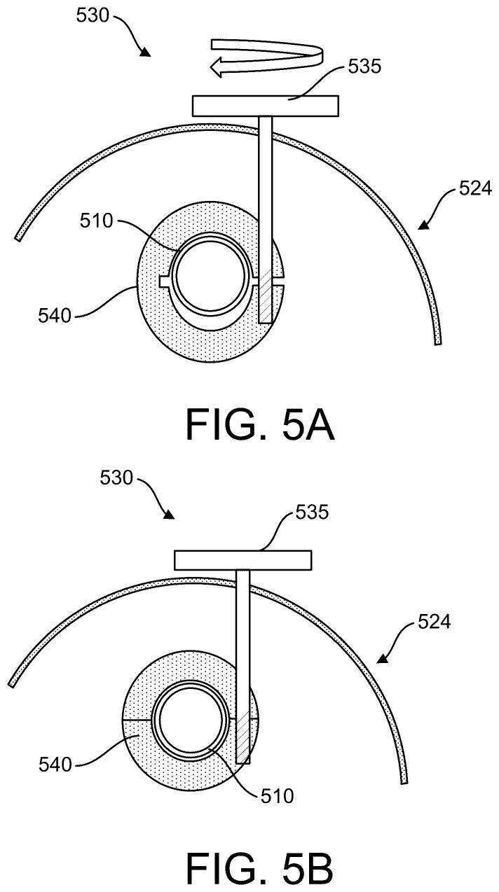

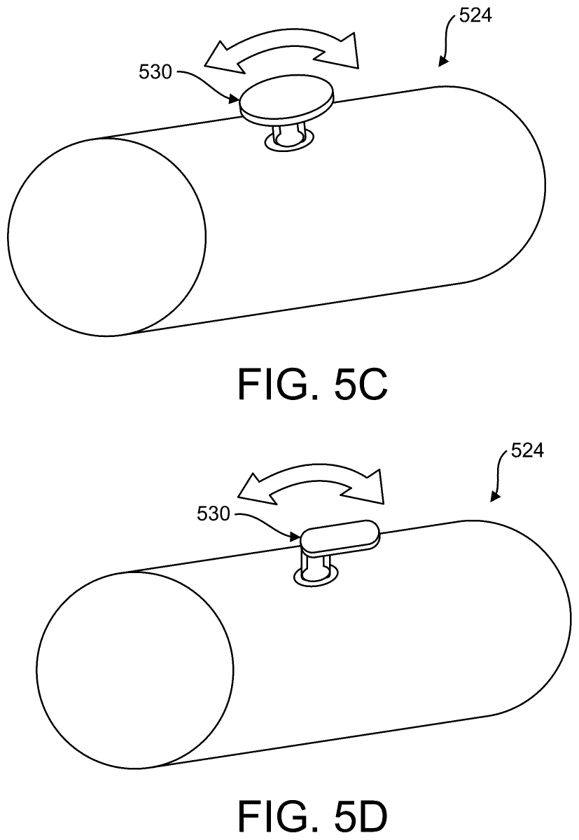

In some embodiments, as shown in FIGS. 5A-5D, the locking mechanism may include a screw collar mechanism 530 having a ring or collar 540 fitting around the perforation device 510, the collar 540 being connected to a distal portion of a screw 535, wherein the proximal portion of the screw 535 protrudes from the dilator handle 524. In one embodiment, the collar 540 may further include friction pads. In certain embodiments, as the screw 535 is rotated, as shown in FIG. 5A, the collar 540, and more specifically the friction pad on the collar 540, may tighten around the perforation device 510, as shown in FIG. 5B, and impede longitudinal movement of the perforation device 510. In certain embodiments, the screw collar mechanism 530 may move from the locked position, FIG. 5B, back to the unlocked position, FIG. 5A, by unscrewing the screw 535 and therefore loosening the collar 540 from around the perforation device 510. In some embodiments, the screw collar mechanism 530 may lock the perforation device 510 in any position on the perforation device 510. In other embodiments, the perforation device 510 may further include at least one or more grooves (not shown), or other alignment features known in the art, along the body of the perforation device 510 and the screw collar mechanism 530 may lock at a set position on the perforation device 510. In certain embodiments, as depicted in FIG. 5C, the screw collar mechanism 530 may be controlled by a user via a wheel on the dilator handle 524; while in other embodiments, as depicted in FIG. 5D, the screw collar mechanism 530 may be controlled by the user via a lever on the dilator handle 524. In still other embodiments, any structure known in the art used to control a screw by a user may be employed.

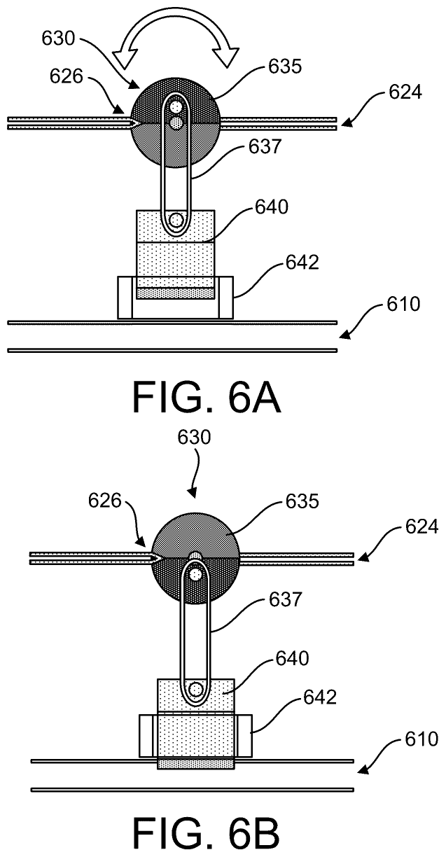

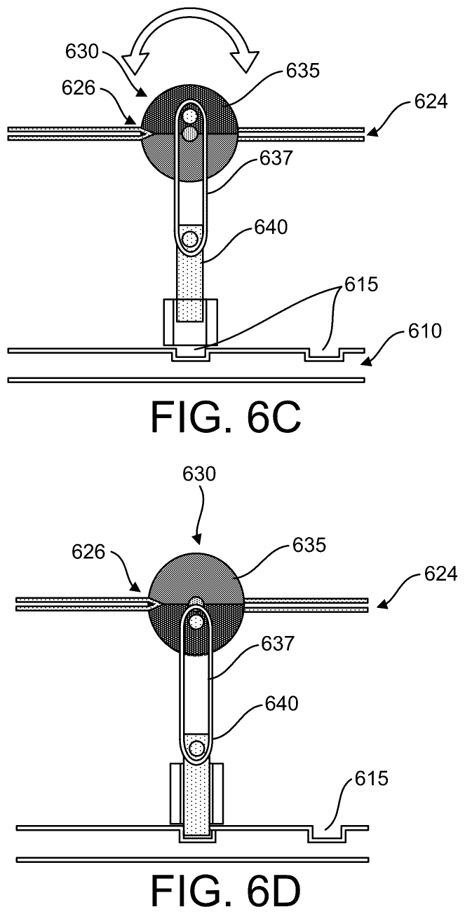

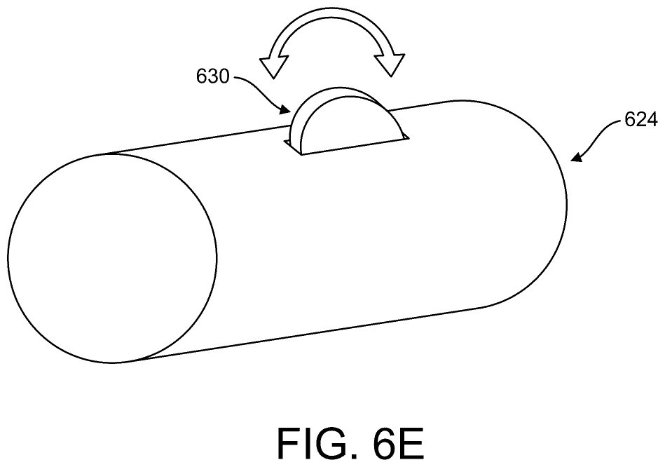

In some embodiments, as shown in FIGS. 6A-6E, the locking mechanism may include a piston mechanism 630 comprising a wheel or axle 635 attached to and protruding from the dilator handle 624. In some embodiments, the piston mechanism 630 is controlled by a user via the wheel 635 positioned on the dilator handle 624. In certain embodiments, the wheel 635 of the piston mechanism 630 may be held in place on the dilator handle 624 via a mating feature 626 on the wheel 635 and the handle 624. In certain embodiments, the mating feature 626 may include at least one or more grooves positioned on of the wheel 635 such that the wheel 635 may lock into the dilator handle 624 in certain positions (i.e., an unlocked position and a locked position). In some embodiments, the piston mechanism 630 further includes a linkage 637 that connects the wheel 635 of the piston mechanism 630 to a piston 640. In still other embodiments, the piston mechanism 630 may further include a piston alignment slot 642 that is attached to the handle 624 and aids in the vertical movement of the piston mechanism 630 from an unlocked position to a locked position. In some embodiments, the piston mechanism 630 may include an unlocked position (i.e., upper position) in which the piston 640 is not in physical contact with the perforation device 610, as shown in FIGS. 6A and 6C, and a locked position (i.e., lower position) in which the piston 640 is in physical communication with the perforation device 610 and may impede longitudinal movement of the perforation device 610, as shown in FIGS. 6B and 6D.

In certain embodiments, as shown in FIGS. 6A-6B, the piston 640 may include a friction pad on the most distal portion of the piston 640. In some embodiments, as the piston mechanism 630 is moved into the locked lower position by the user, the friction pad of the piston 640 may have physical contact with the perforation device 610 thereby preventing longitudinal movement of the perforation device 610. In other embodiments, as depicted in FIGS. 6C-6D, the perforation device 610 may further include at least one or more grooves 615 positioned at set locations on the body of the perforation device 610. In some embodiments, as the piston mechanism 630 is lowered into a locked position, the piston 640 may align with one of the at least one or more grooves 615 on the body of the perforation device 610 thereby impeding the longitudinal movement of the perforation device 610 at a set location. In some embodiments, the piston mechanism 630 may move back to the upper unlocked position by the user rotating the wheel 635 thereby moving the piston 640 upwards and out of the at least one or more grooves 615.

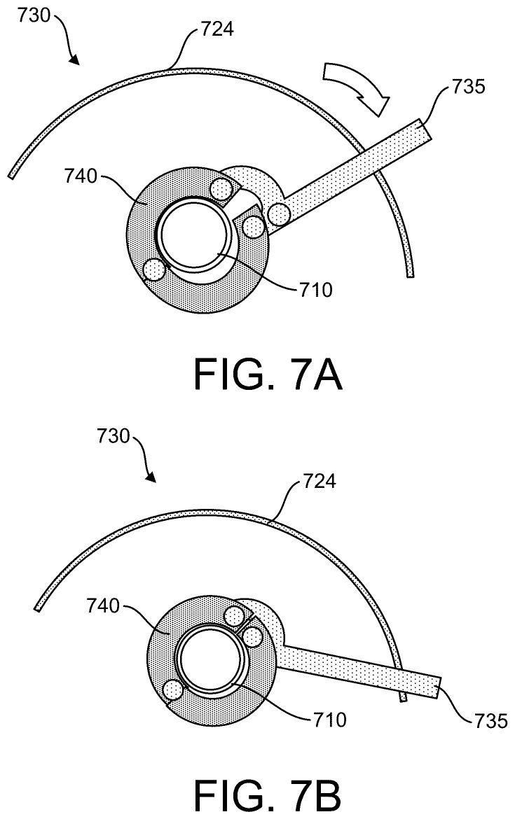

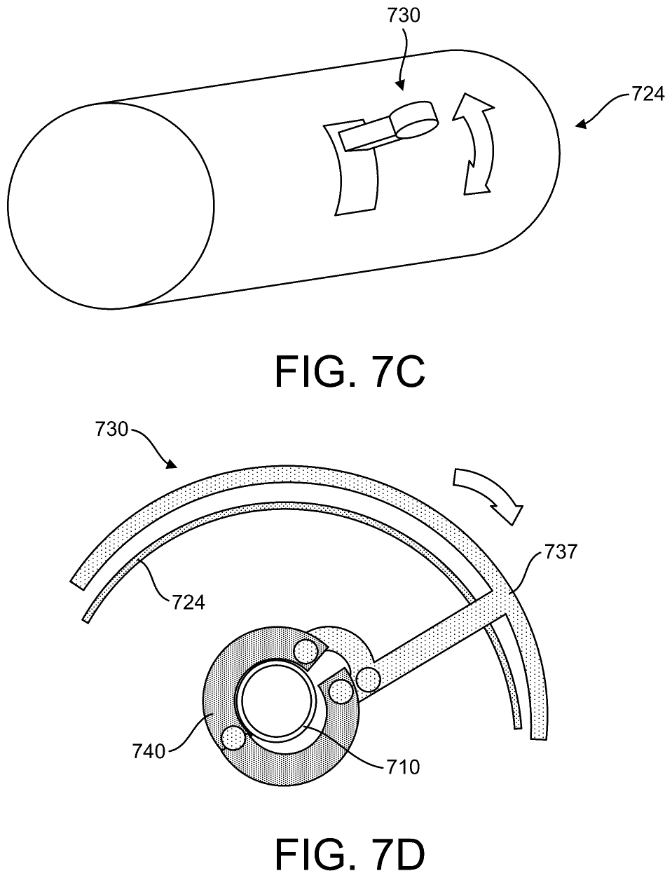

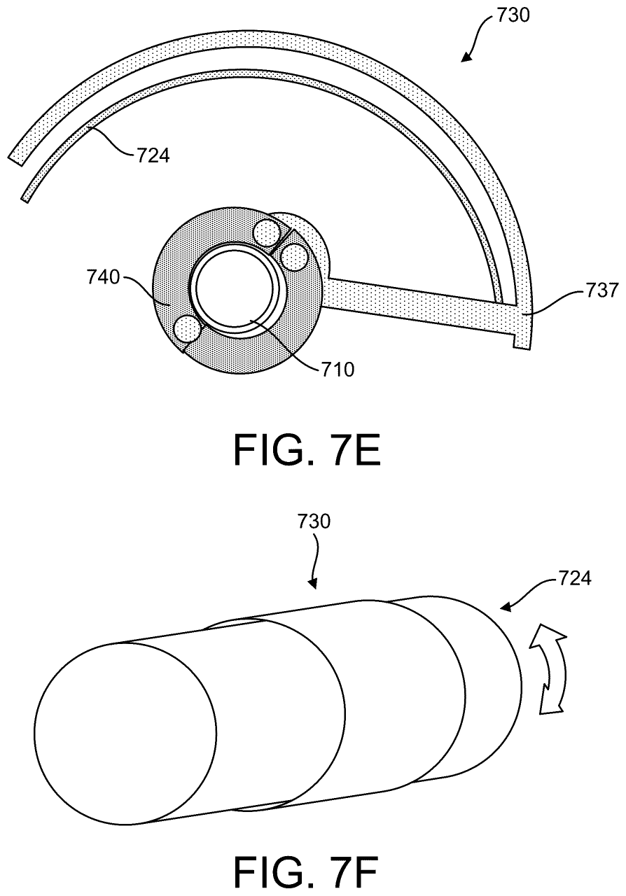

In some embodiments, as shown in FIGS. 7A-7F, the locking mechanism may include a clamp collar mechanism 730 comprising a clamp collar 740 fitting around the perforation device 710. In one embodiment, the clamp collar 740 may further include friction pads. In certain embodiments, the clamp collar 740 may be connected to a distal portion of a switch 735, as shown in FIGS. 7A-7C, or a sleeve 737, as shown in FIGS. 7D-7F. In one example, the proximal portion of the switch 735 may protrude from the dilator handle 724 to be controlled by the user. In another example, as shown in FIGS. 7D-7F, the proximal portion of the sleeve 737 may fully or partially wrap around the dilator handle 724 to be controlled by the user.

In an embodiment, as the switch 735 or the sleeve 737 is actuated by the user, the clamp collar mechanism 730 may move from an unlocked position, FIGS. 7A and 7D, to a locked position, FIGS. 7B and 7E. In certain embodiments, in the locked position the clamp collar 740 tightens around the perforation device 710 thereby impeding longitudinal movement of the perforation device 710. In some embodiments, the clamp collar mechanism 730 may lock the perforation device 710 in any position on the perforation device 710. In other embodiments, the perforation device 710 may further include at least one or more grooves (not shown), or other alignment features known in the art, along the body of the perforation device 710 and the clamp collar mechanism 730 may lock at a set position on the perforation device 710.

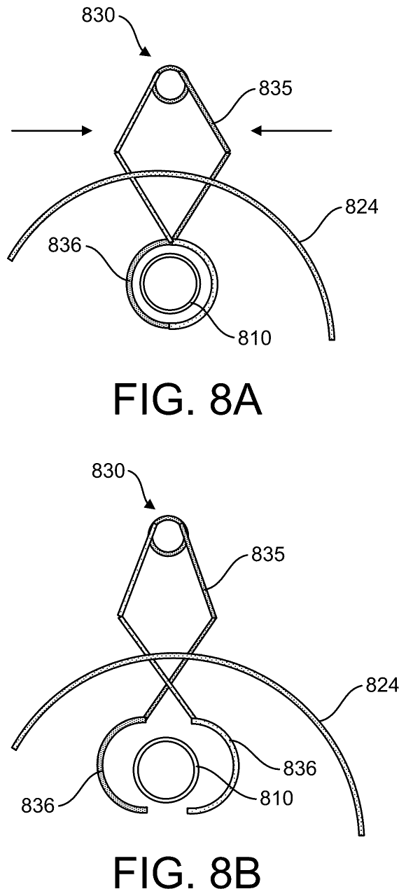

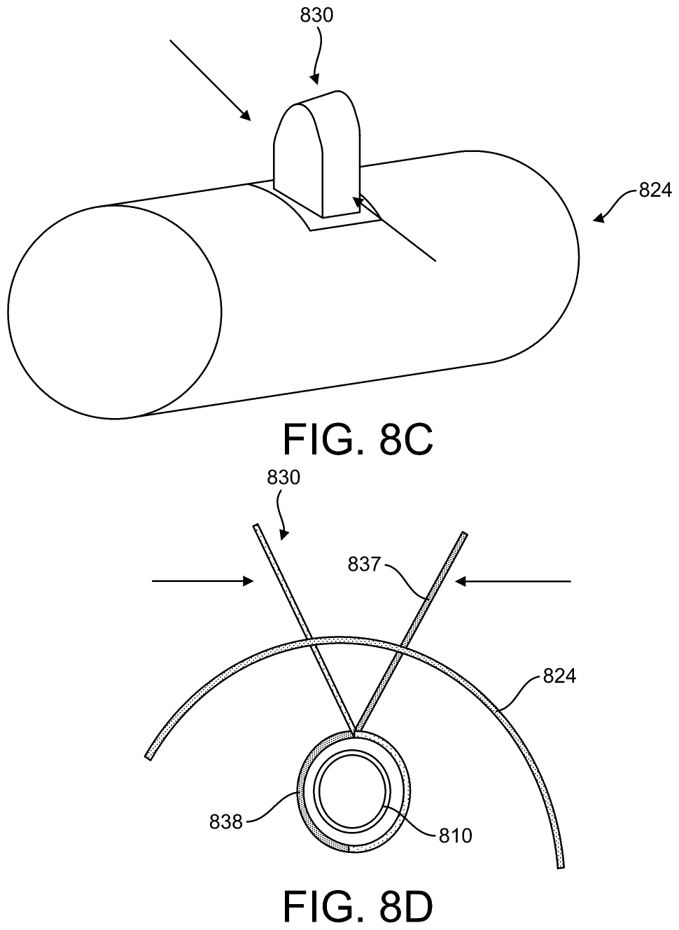

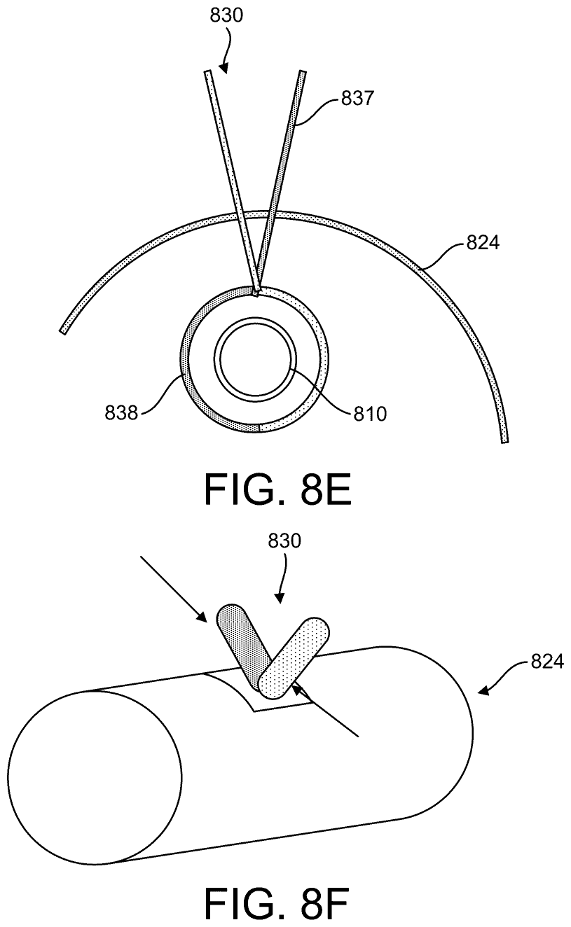

In some embodiments, as shown in FIGS. 8A-8F, the locking mechanism may include a torsion spring mechanism 830. In some embodiments, the spring mechanism 830 may include a spring clamp device 835, as shown in FIGS. 8A-8C. In an embodiment, the spring clamp device 835 may include a distal portion having at least one component 836 bent into a U-shape that may encircle the perforation device 810 connected to a handle that protrudes from the dilator handle 824 and is controlled by the user. In certain embodiments, the spring clamp device 835 may include springs at pivot points on the spring clamp device 835. In some embodiments, as shown in FIGS. 8A-8C, the springs of the spring clamp device 835 are on the outside of the dilator handle 824. In certain embodiments, the default state of the spring clamp device 835 is a locked position, as shown in FIG. 8A, with the distal U-shape components 836 encircling and holding the perforation device 810 in place, thereby impeding longitudinal movement of the perforation device 810. In certain embodiments, to unlock the spring clamp device 835, a user may pinch the handles inward (i.e., closer to each other) to separate the distal U-shape components 836 from each other and from the perforation device 810, thereby allowing the perforation device 810 to move within the dilator lumen if desired.

In other embodiments, the spring mechanism 830 may include a spring clamp device 837, as shown in FIGS. 8D-8F, in which the springs are located inside of the dilator handle 824 and surround the perforation device 810. In an embodiment, the spring clamp device 837 may include a distal portion having a torsion spring 838 encircling the perforation device 810 and connected to a handle that protrudes from the dilator handle 824 and is controlled by the user. In certain embodiments, the default state of the spring clamp device 837 is a locked position, as show in FIG. 8D, with the spring encircling and holding the perforation device 810 in place, thereby impeding longitudinal movement of the perforation device 810. In certain embodiments, to unlock the spring clamp device 837 a user may pinch the handles inward (i.e., closer to each other) to create distance between the spring 838 and the perforation device 810, thereby allowing the perforation device 810 to move within the dilator lumen if desired. In some embodiments, the spring mechanism 830 may lock the perforation device 810 in any position on the perforation device 810. In other embodiments, the perforation device 810 may further include at least one or more grooves (not shown), or other alignment features known in the art, along the body of the perforation device 810 and the torsion spring mechanism 830 may lock at a set position on the perforation device 810.

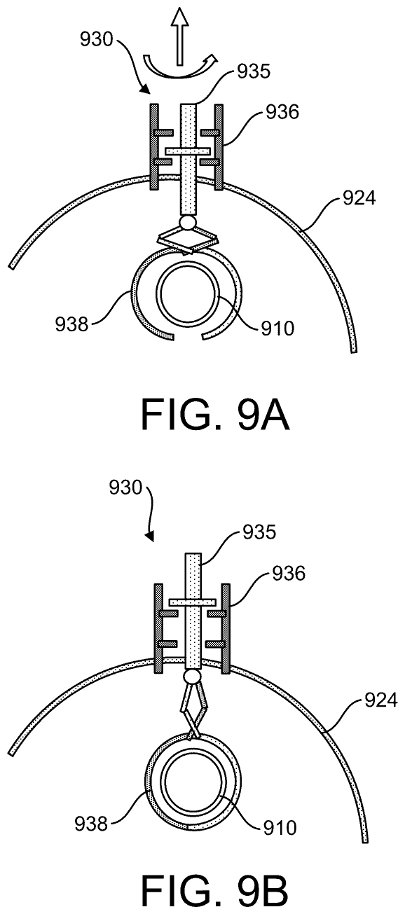

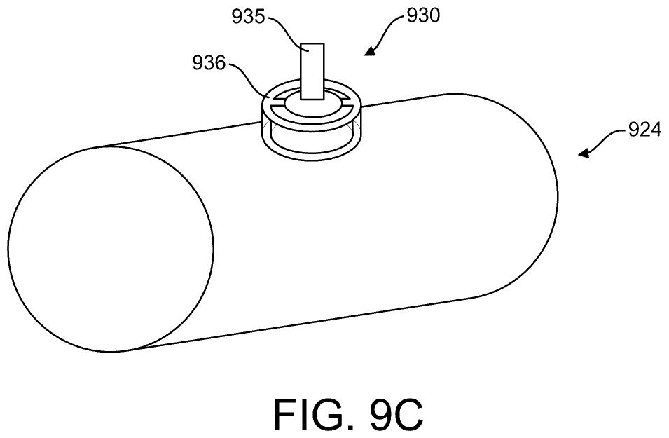

In some embodiments, as shown in FIGS. 9A-9C, the locking mechanism may include a lifting clamp mechanism 930 comprising of a locking interface 936 having a locking feature connected to and protruding from the dilator handle 924. In some embodiments, the lifting clamp mechanism 930 further includes a lifting clamp device having a distal portion comprising of at least one or more clamps 938 that may encircle the perforation device 910, and a proximal portion comprising a handle 935 that protrudes from the dilator handle 924 and is positioned in between the locking interface 936. In certain embodiments, the lifting clamp mechanism 930 includes a lower unlocked position, as shown in FIG. 9A, and an upper locked position, as shown in FIG. 9B. In some embodiments, in the lower unlocked position, the at least one or more clamps 938 may be in an open configuration (i.e., not fully encircling the perforation device 910) and the handle 935 of the lifting clamp device is positioned in between the locking feature of the locking interface 936. In an embodiment, a user may tighten the at least one or more clamps 938 around the perforation device 910 to impede longitudinal movement of the perforation device 910, by twisting the handle 935 so that it is not constrained by the locking feature of the locking interface 936 and pulling the handle 935 upwards to the upper locked position. In an embodiment, by twisting and pulling the handle 935 upwards, the at least one or more clamps 938 may tighten around the perforation device 910, therefore locking the perforation device 910 in place. In some embodiments, to move back to the unlocked position, a user may again twist the handle 935 and move the handle 935 downwards into the locking feature of the locking interface 936 thereby locking the handle 935 into the locking interface 936 and releasing the at least one or more clamps 938 from around the perforation device 910.

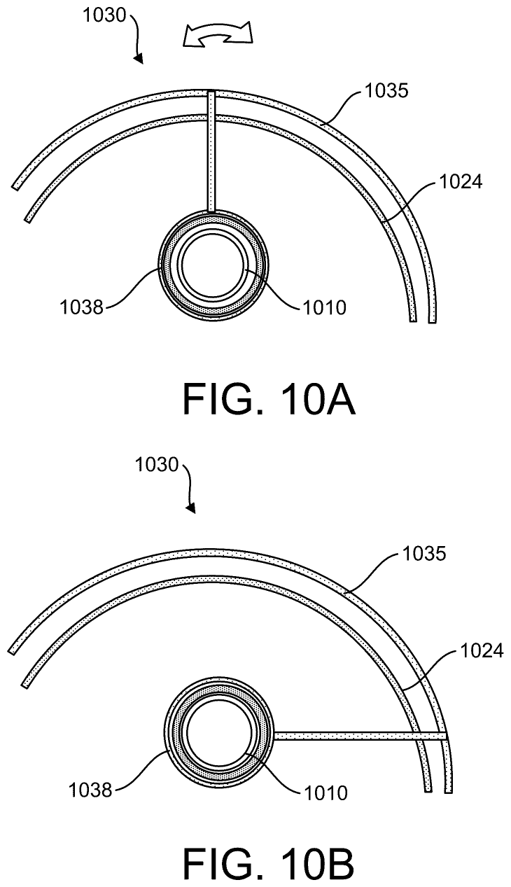

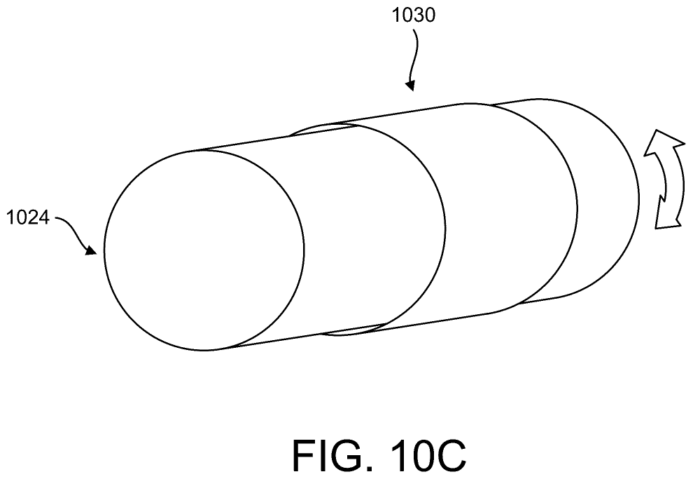

In some embodiments, as shown in FIGS. 10A-10C, the locking mechanism may include a twist lock mechanism 1030 comprising of a distal portion positioned in the dilator lumen having a locking collet 1038 positioned around and encircling the perforation device 1010. In some embodiments, the portion of the locking collet 1038 that may be in physical contact with the perforation device 1010 may further include a friction pad. In certain embodiments, the twist lock mechanism 1030 further includes a handle connected to the locking collet 1038 and also connected to a sleeve 1035 that protrudes from the dilator handle 1024. In certain embodiments, a user may control the twist lock mechanism 1030 via the sleeve 1035.

In an embodiment, the twist lock mechanism 1030 includes an unlocked position, as shown in FIG. 10A, and a locked position, as shown in FIG. 10B. In certain embodiments, a user may move the sleeve 1035 clockwise or counterclockwise thereby twisting and tightening the locking collet 1038 around the perforation device 1010 to impede longitudinal movement of the perforation device 1010. In an embodiment, to move back to the unlocked position the user may twist the sleeve 1035 back in the opposite direction, thereby untwisting the locking collet 1038 from around the perforation device 1010 and allowing free movement of the perforation device 1010. In some embodiments, the twist lock mechanism 1030 may lock anywhere on the perforation device 1010. In other embodiments, the perforation device 1010 may further include at least one or more grooves (not shown), or other alignment features known in the art, along the body of the perforation device 1010 and the twist lock mechanism 1030 may lock at a set location on the perforation device 1010.

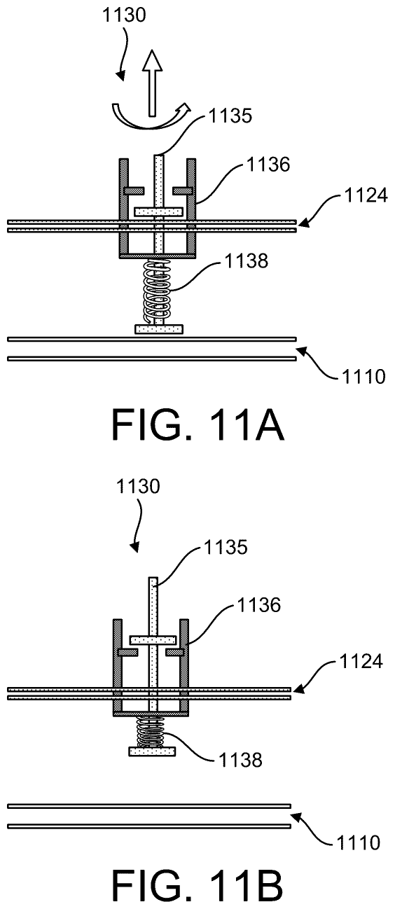

In some embodiments, as shown in FIGS. 11A-11B, the locking mechanism may include a spring mechanism 1130 comprising of a locking interface 1136 having a locking feature connected to and protruding from the dilator handle 1124. In some embodiments, the spring mechanism 1130 further includes a spring device having a distal portion comprising of a spring 1138 and a friction pad that may be in physical contact with the perforation device 1110. In certain embodiments, the spring device further includes a handle 1135 which connects to the friction pad on one end and protrudes from the dilator handle 1124 on the other end for user manipulability. In an embodiment, the spring 1138 is positioned on the distal portion of the handle 1135 and within the dilator lumen. In an embodiment, the spring mechanism 1130 includes a locked position, as shown in FIG. 11A, and an unlocked position, as shown in FIG. 11B. In certain embodiments, the default state of the spring mechanism 1130 is in the locked position with the spring 1138 in a relaxed or stretched state and in physical communication with the perforation device 1110 thus impeding longitudinal movement of the perforation device 1110. In some embodiments, to move to the unlocked position, the user may twist the handle 1135 so that it is not constrained by the locking feature of the locking interface 1136 and pulling the handle 1135 upwards to compress the spring 1138 and remove the contact between the friction pad of the spring mechanism 1130 and the perforation device 1110. In some embodiments, the spring mechanism 1130 may lock anywhere on the perforation device 1110.

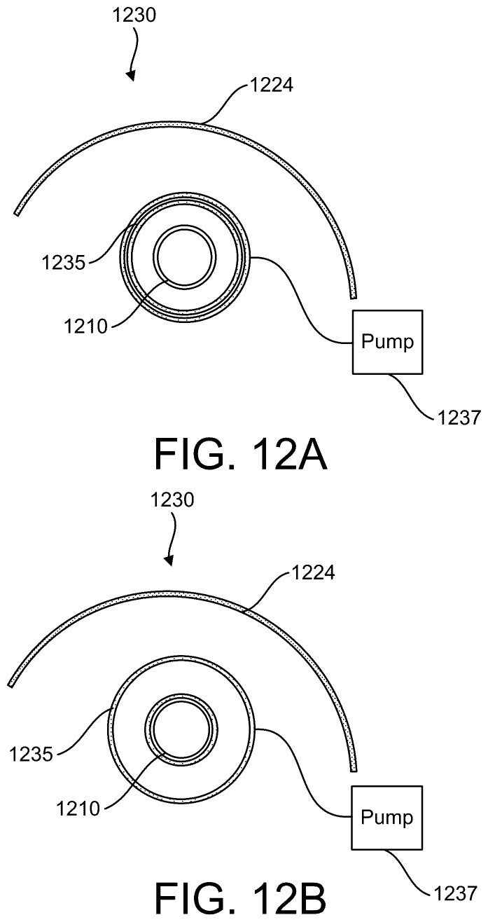

In some embodiments, as shown in FIGS. 12A-12B, the locking mechanism may include a pumping mechanism 1230 comprising a pump 1237 positioned outside of the dilator handle 1224 for user manipulability and connected to a balloon 1235 positioned within the dilator lumen. In certain embodiments, the balloon 1235 may include friction pads and may be in physical contact with the perforation device 1210. In an embodiment, the pumping mechanism 1230 may include an unlocked position in which the balloon 1235 is not in physical communication with the perforation device 1210, as shown in FIG. 12A, and a locked position in which the balloon 1235 is in physical communication with the perforation device 1210, as shown in FIG. 12B. In some embodiments, to impede longitudinal movement of the perforation device 1210, a user may inflate the balloon 1235 via the pump 1237 positioned outside of the dilator handle 1224. In an embodiment, the inflated balloon 1235 may surround and tighten around the puncture device 1210 to hold the position of the puncture device 1210. In some embodiments, the pumping mechanism 1230 may lock anywhere on the perforation device 1210. In an embodiment, to move to the unlocked position, a user may deflate the balloon 1235 via the pump 1237 to release the hold the balloon 1235 has around the perforation device 1210.

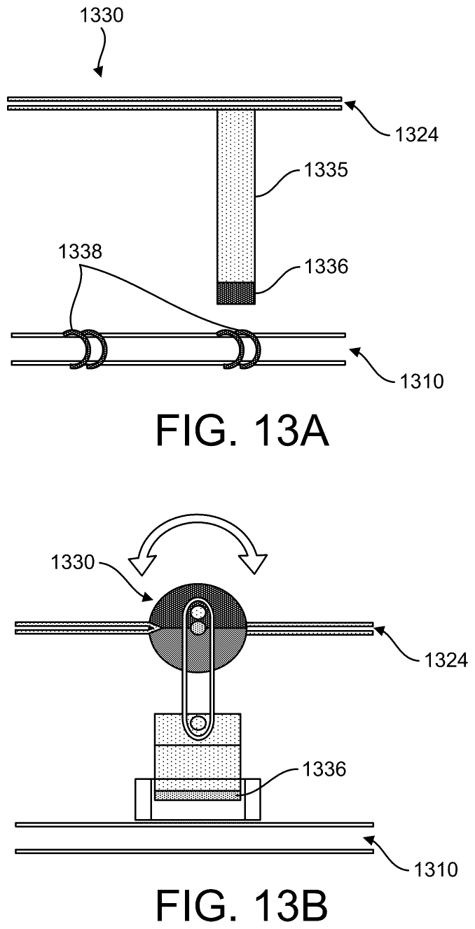

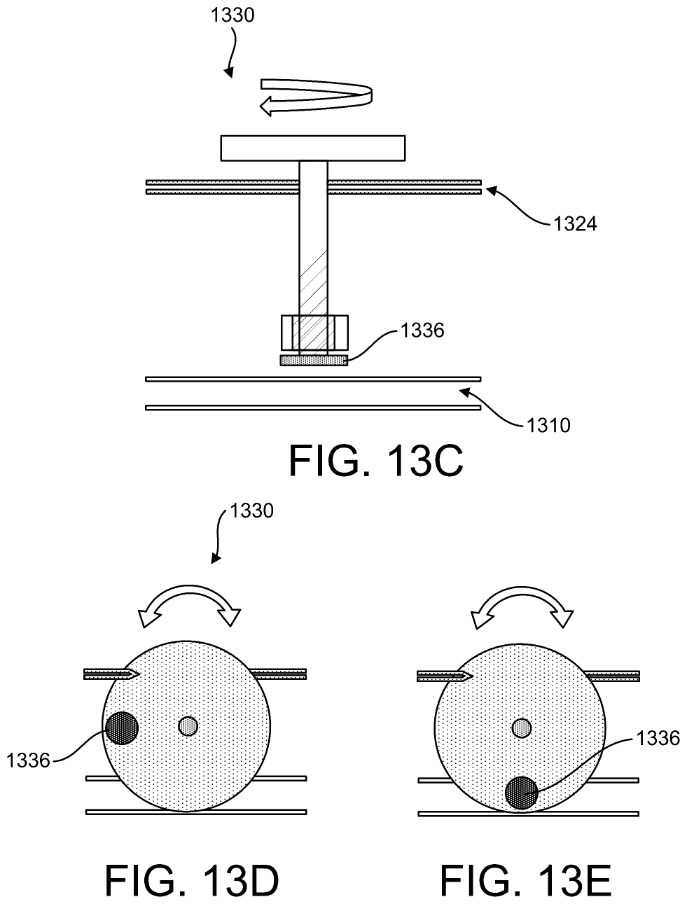

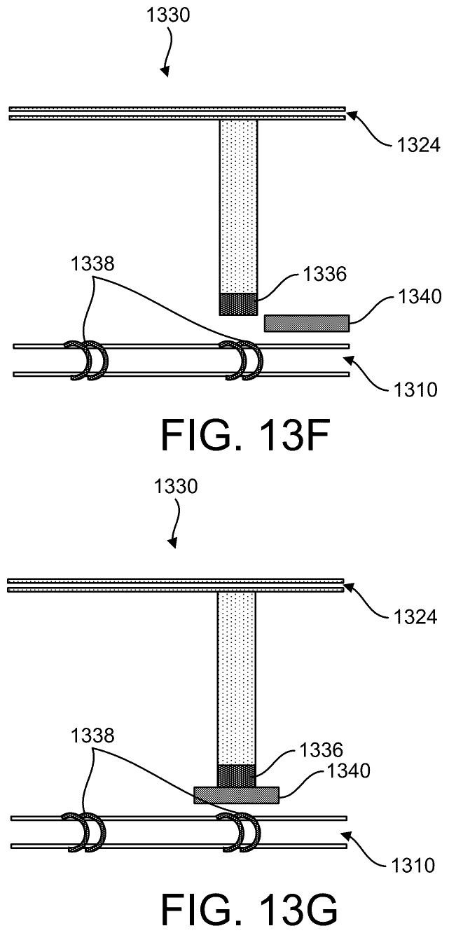

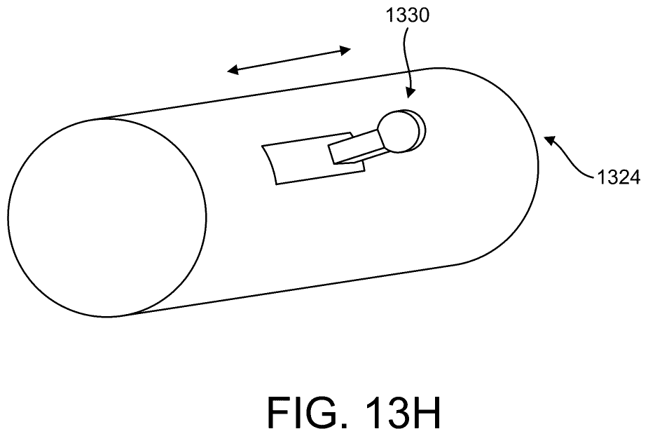

In some embodiments, as shown in FIGS. 13A-13H, the locking mechanism may include a magnetic mechanism 1330. In an embodiment, the magnetic mechanism 1330 may include an unlocked position in which the magnet is not in physical communication with the perforation device 1310 and a locked position in which the magnetic mechanism 1330 is in physical communication with the perforation device 1310. As shown in FIG. 13A, in an embodiment, the magnetic mechanism 1330 may include a fixed magnet in which the perforation device 1310 includes at least one or more magnetic areas 1338 at specific locations on the perforation device 1310. In some embodiments, the at least one or more magnetic areas 1338 may be attracted by an opposite pole magnet 1336 that is connected to a handle 1335 that is positioned within the dilator lumen and is connected to an inside portion of the dilator handle 1324. In an embodiment, as the perforation device 1310 advances laterally through the dilator lumen, the opposite pole magnet 1336 may attract one of the at least one or more magnetic areas 1338 on the perforation device 1310. In an embodiment, the opposite pole magnet 1336 is strong enough to hold the position of the perforation device 1310 while not strong enough that the magnet 1336 cannot be disconnected from the perforation device 1310. As shown in FIGS. 13F-13G, in an embodiment, the magnetic mechanism 1330 may include a shielded magnet mechanism in which a magnet shield 1340 may be moved in front of the fixed magnet 1136 via a switch, as shown in FIG. 13H, or a slide mechanism, to unlock the perforation device 1310. In an embodiment, the magnetic mechanism 1330 is in a locked position when the shield 1340 is removed from in front of the opposite pole magnet 1336 and the opposite pole magnet 1336 can attract one of the at least one or more magnetic areas 1338 on the perforation device 1310.

In an embodiment, as shown in FIGS. 13B-13E, the magnetic mechanism 1330 may include an actuated magnet in which the magnetic mechanism may lock at a specific location on the perforation device 1310 or anywhere along the length of the perforation device 1310. In certain embodiments, the perforation device 1310 may include magnets at fixed positions on the perforation device 1310, while in other embodiments, a majority of the perforation device may be magnetic. In an embodiment, to unlock the magnetic mechanism 1310, a user may move the magnet away from the puncture device 1310. In an embodiment, as shown in FIG. 13B, the magnetic mechanism 1330 may include a piston mechanism, as described above, including a magnet 1336 at the most distal portion of the piston. In another embodiment, as shown in FIG. 13C, the magnetic mechanism 1330 may include a screw mechanism, as discussed above, including a magnet 1336 at the most distal portion of the screw. In another embodiment, as shown in FIG. 13D-13E, the magnetic mechanism 1330 may include a wheel that may be rotated with a magnet 1336 on one side of the wheel so that when the magnet is in communication with the perforation device 1310, the perforation device 1310 is prevented from moving laterally within the dilator lumen.

Furthermore, in some embodiments, as discussed above, the locking mechanism may work congruently with the indicator mechanism and the needle advancement mechanism discussed above. Thus, in some embodiments, once the perforation device is advanced by the needle advancement mechanism and is positioned at the desired perforation length by the aid of the indicator mechanism, the perforation device may then be locked in position by the locking mechanism. In an embodiment, locking the perforation device in place may allow a hand of a user to be free to feed an anchor wire into the dilator lumen. In certain embodiments, this allows a change in workflow to eliminate the step of dilating the septum with the perforation needle protruded, which is the step that generally risks tamponade. Thus, in some embodiments, once the device is positioned on the septum, the perforation needle may protrude out of the dilator and position against the septum. In an embodiment, the needle may then puncture the tissue and create access to the left atrium. In an embodiment, the needle may then be locked using the locking mechanism and a guidewire may be passed through the needle within the dilator lumen. In an embodiment, with the guidewire holding the position originally created by the perforation needle, the needle may then be unlocked, retracted and the dilator can cross over the guidewire.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. An access system for perforating a tissue within a heart, the system comprising:
a perforation device having a perforation device body including a proximal portion and a distal portion terminating in a distal tip;
a dilator comprising an elongated body having a body length, a proximal end and a distal end portion terminating in a distal tip, a handle having a handle length and connected to the proximal end of the elongated body, and a dilator lumen extending through the handle and the elongated body and dimensioned to slidingly receive the perforation device, wherein the body length and the handle length together define a dilator length;
an indicator mechanism positioned on the dilator indicating a protrusion length of the perforation device; and
a locking mechanism located on the dilator having an unlocked position and a locked position, wherein in the locked position the locking mechanism is in physical communication with the perforation device, and wherein the locking mechanism is configured to impede longitudinal movement of the perforation device within the dilator lumen.

2. The system of claim 1, further comprising a needle advancement mechanism positioned on the dilator, wherein the needle advancement mechanism is configured to interact with the perforation device and advance the perforation device longitudinally within the dilator lumen.

3. The system of claim 2, wherein:
the perforation device includes at least one or more grooves located on the perforation device body;
the needle advancement mechanism is a knob having a top portion protruding from the dilator and a bottom portion positioned within the dilator lumen; and
when the knob aligns with one of the at least one or more grooves within the perforation device body, a user longitudinally advances the top portion of the knob across the dilator thereby advancing the perforation device longitudinally within the dilator lumen.

4. The system of claim 2, wherein the needle advancement mechanism is a dial having a traction element, and wherein friction applied by the dial to the perforation device advances the perforation device longitudinally within the dilator lumen.

5. The system of claim 2, wherein the needle advancement mechanism is an accessory device.

6. The system of claim 1, wherein the protrusion length is measured from the distal tip of the dilator to the distal tip of the perforation device.

7. The system of claim 1, wherein the indicator mechanism includes an indicator, and wherein the indicator is at least one of a visual, a tactile or an auditory indicator.

8. The system of claim 7, wherein the indicator includes at least one or more markings on the handle of the dilator representing the protrusion length of the perforation device.

9. The system of claim 7, wherein the indicator includes an indicator light that communicates the protrusion length of the perforation device.

10. The system of claim 1, wherein:
the locking mechanism includes a friction pad on a portion of the locking mechanism in contact with the perforation device; and
in the locked position, the pressure applied by the friction pad creates resistance on the perforation device thereby impeding longitudinal movement of the perforation device.

11. The system of claim 1, wherein:
the perforation device includes at least one or more grooves positioned on the perforation device body; and
in the locked position the locking mechanism aligns with one of the at least one or more grooves on the perforation device body to impede longitudinal movement of the perforation device.

12. An access device for perforating a tissue within a heart, the device comprising:
a perforation needle having a perforation needle body including a proximal portion and a distal portion terminating in a distal tip;
a dilator comprising an elongated body having a body length, a proximal end and a distal end portion terminating in a distal tip, a handle having a handle length and connected to the proximal end of the elongated body, and a dilator lumen extending through the handle and the elongated body and dimensioned to slidingly receive the perforation device, wherein the body length and the handle length together define a dilator length;
an indicator mechanism positioned on the dilator indicating a protrusion length of the perforation needle;
a needle advancement mechanism positioned on the dilator configured to move the perforation needle longitudinally within the dilator lumen; and
a locking mechanism located on the dilator having an unlocked position and a locked position, wherein in the locked position the locking mechanism is in physical communication with the perforation needle, and wherein the locking mechanism is configured to impede longitudinal movement of the perforation needle within the dilator lumen.

13. The device of claim 12, wherein the protrusion length is measured from the distal tip of the dilator to the distal tip of the perforation device.

14. The device of claim 12, wherein the indicator mechanism includes an indicator, and wherein the indicator is at least one of a visual, a tactile or an auditory indicator.

15. The device of claim 12, wherein:
the locking mechanism includes a friction pad on a portion of the locking mechanism in contact with the perforation device; and
in the locked position, the pressure applied by the friction pad creates resistance on the perforation device thereby impeding longitudinal movement of the perforation device.

16. The device of claim 12, wherein:
the perforation device includes at least one or more grooves positioned on the perforation device body; and
in the locked position the locking mechanism aligns with one of the at least one or more grooves on the perforation device body to impede longitudinal movement of the perforation device.

17. The device of claim 12, wherein:
the locking mechanism is a screw inserted into the handle of the dilator, the screw having a top portion protruding from the handle and a bottom portion positioned within the dilator lumen;
the top portion of the screw includes a wheel or a lever to be manipulated by a user; and
in the locked position the screw is in physical communication with the perforation device and impedes longitudinal movement of the perforation device.

18. The device of claim 12, wherein the locking mechanism is a collar, and wherein in the locked position the collar tightens around the perforation device to impede longitudinal movement of the perforation device.

19. The device of claim 12, wherein:
the locking mechanism is a spring positioned within the handle of the dilator, the spring having a top portion protruding from handle and a bottom portion positioned within the dilator lumen;
in the locked position the spring is in a neutral state and in physical communication with the perforation device; and
in the unlocked position the spring is in a compressed state and not in physical communication with the perforation device.

20. A method for perforating a tissue within a heart, the method comprising:
providing a perforation device having a perforation device body including a proximal portion and a distal portion terminating in a distal tip;
providing a dilator comprising an elongated body having a body length, a proximal end and a distal end portion terminating in a distal tip, a handle having a handle length and connected to the proximal end of the elongated body, and a dilator lumen extending through the handle and the elongated body and dimensioned to slidingly receive the perforation device, wherein the body length and the handle length together define a dilator length;
advancing an indicator mechanism positioned on the dilator indicating a protrusion length of the perforation device; and
using a locking mechanism located on the dilator having an unlocked position and a locked position, wherein in the locked position the locking mechanism is in physical communication with the perforation device, and wherein the locking mechanism is configured to impede longitudinal movement of the perforation device within the dilator lumen.
